(19) United States
(12) Patent Application Publication
(10) Pub. No.: US 2018/0249939 A1
(43) Pub. Date: Sep. 6, 2018

(54) STRESS DETECTION AND MANAGEMENT SYSTEM

(71) Applicant: Intel Corporation, Santa Clara, CA (US)

(72) Inventor: Jinshi Huang, Fremont, CA (US)

(21) Appl. No.: 15/450,443

(22) Filed: Mar. 6, 2017

(51) Int. Cl. A61B 5/16; A61B 5/0402; A61B 5/00; A61B 5/0205

(52) U.S. Cl. CPC A61B 5/165; A61B 5/0402; A61B 5/7264; A61B 5/0533; A61B 5/726; A61B 5/742; A61B 5/6897; A61B 5/0205

(57) ABSTRACT

The stress detection and management includes a human interface device having a mouse portion to generate cursor control signals and a stress detection portion. The stress detection portion includes a plurality of sensor probes to detect a user's electrical skin response to stress. A sensor is coupled to the plurality of sensor probes to generate a voltage indicative of the skin response to stress. A neural network, coupled to the sensor, generates a stress classification indication based on the voltage indicative of the skin response and pre-training of the neural network with stress indications. The neural network is retrained by comparing the baseline stress classification with the user inputs, using heuristic rules.

### TECHNICAL FIELD

Embodiments described herein pertain in general to stress detection and management and in particular to using an artificial neural network in combination with sensors in a computer mouse device to detect and manage personal stress levels.

### BACKGROUND

Workplace stress may be the cause of significant costs to employers in terms of employee turnover, missed work by employees, insurance costs, and workers' compensation. Workers who report that they are stressed during their work day may incur health costs that are substantially higher than workers who are not stressed. Seven out of ten deaths each year are from chronic diseases (e.g., heart disease) in which stress may be a contributing factor.

### BRIEF DESCRIPTION OF THE DRAWINGS

FIG. 1 illustrates a diagram of a human interface device including sensors, according to various embodiments.

FIG. 2 illustrates a block diagram of the human interface device including a stress detection portion, according to various embodiments.

FIG. 3 illustrates a flowchart of a method for signal processing and extraction of heartbeat features, according to various embodiments.

FIG. 4 illustrates various heart rate variabilities, according to various embodiments.

FIG. 5 illustrates a flowchart of a method for signal processing and extraction of Galvanic skin response (GSR) features, according to various embodiments.

FIG. 6 illustrates parameters of a GSR signal, according to various embodiments.

FIG. 7 illustrates a block diagram of an artificial neural network in a self-learning embodiment, according to various embodiments.

FIG. 8 illustrates a flow diagram of a method for pre-training the neural network, according to various embodiments.

FIG. 9 illustrates a block diagram of a stress management system, according to various embodiments.

FIG. 10 illustrates a diagram of a stress management tool, according to various embodiment.

FIG. 11 illustrates a block diagram of a computer system, according to various embodiments.

FIG. 12 illustrates a flowchart of a method for stress detection and management, according to various embodiments.

### DETAILED DESCRIPTION

Due to significant problems resulting from workplace stress, it is desirable to detect, track, and attempt to reduce this stress. Clinical research has shown that one of the most reliable indicators of stress is the heart rate variability (HRV). HRV is the variation in the time interval between one heartbeat and the next. When HRV levels are relatively low, this may be an indication of greater stress and lower resiliency. When HRV levels are relatively high, this may be an indication of less stress and higher resiliency.

User stress may also be monitored by measuring the skin conductance (SC) or Galvanic skin response (GSR). GSR includes two main components: Skin Conductance Level (SCL), related to the certain amount of continuity over time (tonic value), and Skin Conductance Response (SCR), which stands for the change in SC within a short period of time as a reaction toward a discrete stimulus. Studies have shown that SCR may be a good measure of emotional response such as stress, anxiety, fear and anger.

Existing devices used to monitor personal stress range from high-end medical devices (e.g. Empatica E4) to low-cost wearable devices for fitness (e.g. Jawbone® Up3). These devices may communicate with stress management tools including apps on mobile phones and/or web-based programs managed by professionals. Most of these devices are either operated in a medical facility or disconnected from the work environment, rendering themselves unfeasible as tools for workplace stress management.

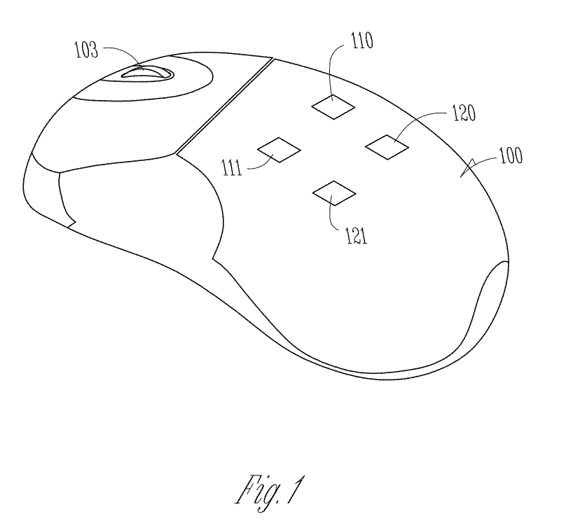

In the present embodiments, stress detection and management capabilities are integrated into one of the most pervasive human interface devices in the workplace, the computer mouse. FIG. 1 illustrates an embodiment of such a device.

FIG. 1 illustrates a diagram of a human interface device including sensors, according to various embodiments. The human interface device comprises a stress detection device integrated with a computer mouse.

The term “computer mouse” may be defined as any human-computer interface device that is hand controlled and translates user hand or finger/thumb movement into two-dimensional cursor movement on a computer monitor. The definition of computer mouse may include a device that detects two-dimensional motion of the device relative to a surface or a trackball device that detects two-dimensional movement of a ball within the device. The mouse portion may thus be a cursor control portion.

The mouse may include one or more buttons 103 for selection by the cursor of text and or images on the monitor as well as a movement tracking device (e.g., track ball, light emitting diode (LED)) (not shown) for tracking movement of the device.

The stress detection portion integrated into the mouse body 100 includes two probes 120, 121 for the electrocardiogram (ECG) sensor device and another two probes 110, 111 for the GSR sensor device. One probe of each set of probes 110, 111, 120, 121 may be a positive probe and the other probe may be a negative probe. Both sets of probes 110, 111, 120, 121 may be located on top of the mouse surface of the mouse body 100 such that they are in contact with the surface of the user's palm when the user's hand is resting on the mouse body 100 in order to detect a user's electrical skin response to stress. The number and locations of the ECG and GSR probes integrated into the mouse body 100 are for purposes of illustration only. Other embodiments may use different numbers and/or locations for these probes.

The stress detection device may include other types of probes besides the ECG and GSR probes. For example, another embodiment may include a temperature probe for measuring skin temperature.

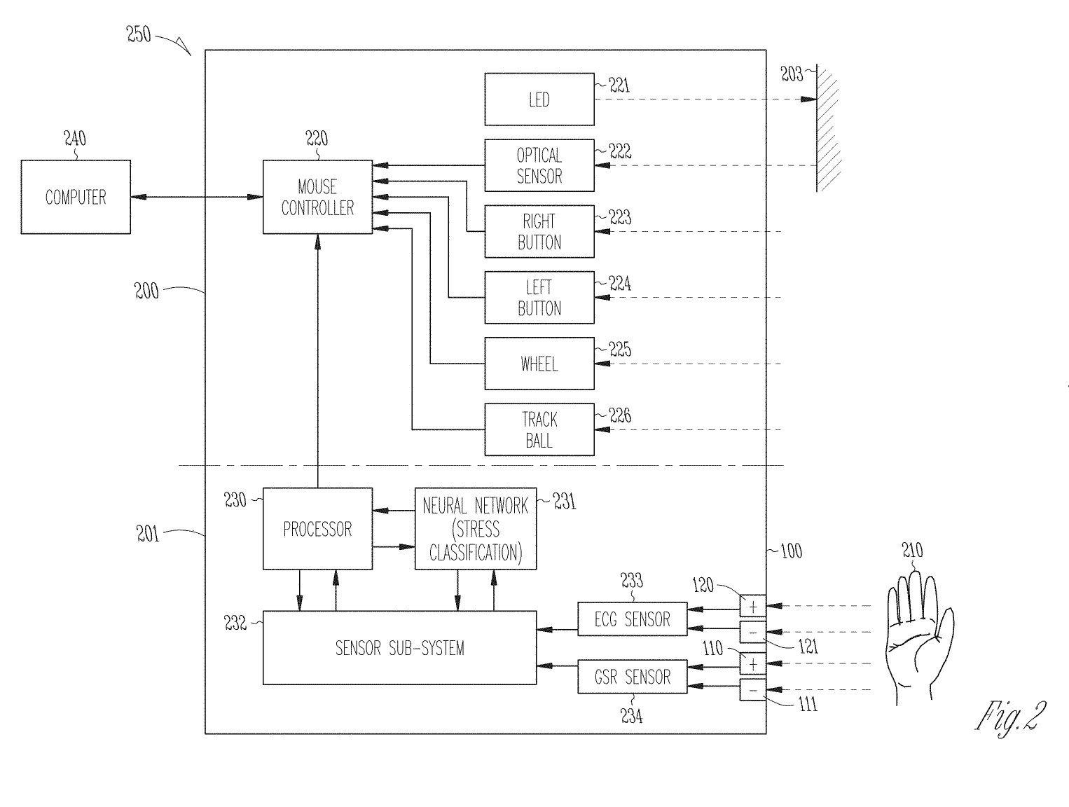

FIG. 2 illustrates a block diagram of the human interface device 250 including the stress detection portion 201, according to various embodiments. The human interface device 250 includes a cursor control portion 200 and the stress detection portion 201. The human interface device 250 may also represent portions of a laptop, tablet computer, or desktop computer having a stress detection portion 201.

The mouse portion 200 includes a motion tracking element such as an LED 221 that projects a light onto a surface 203. The light movement may then be tracked by an optical sensor 222 to detect movement of the mouse and, thus, move the cursor on the monitor. In another embodiment, the mouse portion 200 includes a track ball (e.g., track ball and optical trackball sensor) 226 whose movement may be detected by the LED 221 and trackball sensor 226 to move the cursor on the monitor.

The mouse portion 200 further includes one or more buttons 223, 224. For example, one mouse may have a right button 223 and a left button 224. Another mouse may have only one button 223. The mouse portion 200 may also include a scroll wheel 225 for moving objects and/or scroll bars on the monitor.

The mouse controller 220 is coupled to the optical sensor 222, the one or more buttons 223, 224, the scroll wheel 225, or, in a trackball embodiment, to the track ball sensor 226. The mouse controller 220 comprises a processor or other control circuitry to output cursor control signals to the computer based on the activation of the one or more buttons 223, 224, the wheel 225, the movement of the mouse, or movement of the track ball 226.

The stress detection portion 201 includes the various sensor probes 110, 111, 120, 121 that are located on the mouse surface 100. This diagram shows a user's hand 210 whose palm would be in contact with the probes 110, 111, 120, 121 during operation of the mouse. This is for purposes of illustration only as the sensor probes 110, 111, 120, 121 may be located such that one set (e.g., sensor probes 110, 111) are under one user's hand and another set (e.g., sensor probes 120, 121) are under the other user's hand. The ECG sensor probes 120, 121 are coupled to an ECG sensor 233. The GSR sensor probes 110, 111 are coupled to a GSR sensor 234.

The sensor probes 110, 111, 120, 121 may also be located in different locations of the human interface device 250. For example, if the human interface device 250 is a tablet computer, the sensor probes 110, 111, 120, 121 may be located on a top side, a bottom side, or both sides of the tablet. The stress detection portion 201 may be located on top of a mouse, on a palm rest of a lap top computer, the back of a tablet computer, or the track pad of any computer.

The ECG sensor 233 generates a voltage that is indicative of an electrical potential generated by electrical activity in cardiac tissue. Current flow, in the form of ions, signals contraction of cardiac muscle fibers leading to the heart's pumping action. The ECG sensor probes 120, 121 detect this ion flow in the user's skin and the ECG sensor 233 generates a representative voltage that indicates when a heartbeat has occurred by the presence of the ion flow. The ECG sensor 233 also measures the time between each heartbeat to generate both the heart rate and the inter-beat interval. The inter-beat interval may then be used to generate the HRV.

The GSR sensor 234 generates an indication of a user's electrodermal activity (EDA). EDA is a property of the human body that causes a continuous variation in the electrical characteristics of the skin when an external voltage is applied by one of the sensor probes 110, 111 and received by the other sensor probe 111, 110. A GSR signal includes two components: skin conductance level (SCL) and skin conductance response (SCR). SCL represents the particular change in skin conductance over time (e.g., tonic value). SCR represents the change in skin conductance within a relatively short period of time as a reaction toward a discrete stimulus (e.g., phase value).

Skin resistance has been shown to vary with the state of sweat glands in the skin. Sweating is controlled by the sympathetic nervous system. Thus, skin conductance may be used as an indication of psychological or physiological arousal. If the sympathetic branch of the autonomic nervous system is highly aroused, then sweat gland activity also increases, which in turn increases skin conductance. In this way, skin conductance can be a measurement of emotional and sympathetic responses. SCR may be characterized by parameters such as amplitude (SCR amp), latency of response onset (SCR lat), rise time of the response peak (SCR ris.t), and the half time of recovery time (SCR rec. ½). These parameters are shown in FIG. 6 and discussed subsequently.

The ECG sensor 233 and the GSR sensor 234 are coupled to a sensor subsystem 232. The sensor subsystem 232 may include analog-to-digital converter (ADC) circuitry that converts the analog voltages, as measured by the sensor probes 110, 111, 120, 121 and sensors 233, 234, into digital representations of those voltages. The sensor subsystem 232 may include other circuitry (e.g., filters, amplifiers) to condition the measured signal either prior to the ADC or after the ADC.

The sensor subsystem 232 is coupled to a processor (e.g., microprocessor, control circuitry, controller) 230 and a neural network 231. The processor 230 is also coupled to the neural network 231 and the mouse controller 220.

The processor 230 provides control for the stress detection portion 201 of the human interface device 250. The processor 230 interfaces between the sensor subsystem 232 and the mouse controller 220 as well as the neural network 231 and the mouse controller 220.

The neural network 231 generates a stress classification of the user's stress based on the measurement responses (e.g., ECG, GSR) and a pre-trained parameters of stress indications. The stress measurements and/or classification are transmitted to a computer 240 over a wired (e.g., USB) or wireless (e.g., Bluetooth) channel.

The stress measurements and/or stress classification may be integrated into productivity tools, executed on the computer 240, such as a calendar to manage the stress. Proper actions (which can be customized individually), such as reminders for a coffee break or meditation, can be taken as the worker's stress reached certain levels.

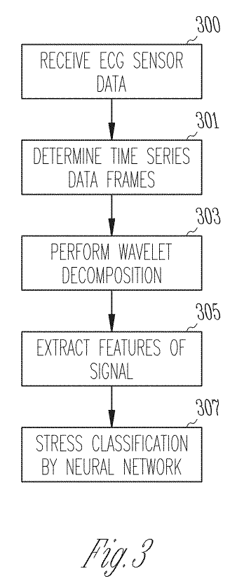

FIG. 3 illustrates a flowchart of a method for signal processing and extraction of heartbeat signals, according to various embodiments. This method may be executed by the ECG sensor 233, the sensor subsystem 232, or both.

In the illustrated embodiment, the heartbeat signals are the ECG data. The step of digitizing the ECG sensor data is not shown in the flowchart since it can be performed at any part of the method. For example, the entire method may be performed on a digitized ECG signal after receipt of an analog ECG signal from the sensor probes 120, 121, or the digitization may be performed anywhere between any operations.

In block 300, the ECG data is received from the sensor probes 120, 121. In block 301, the time series data frames are determined. One frame is typically of a fixed number of ECG samples (for example, 1024) that are that are meaningful and convenient for the subsequent processing (such as wavelet decomposition).

In block 303, wavelet decomposition is performed. The wavelet decomposition converts the signal from the time domain to the frequency domain in order to observe the shape of the signal to determine the features (e.g., heartbeat indications). The wavelet decomposition may be a discrete wavelet transform operation in which the wavelets are discretely sampled. The wavelet decomposition captures both the frequency and location in time of the features.

In block 305, the features found in the wavelet decomposition are extracted. This step determines the timing of the heartbeat indications.

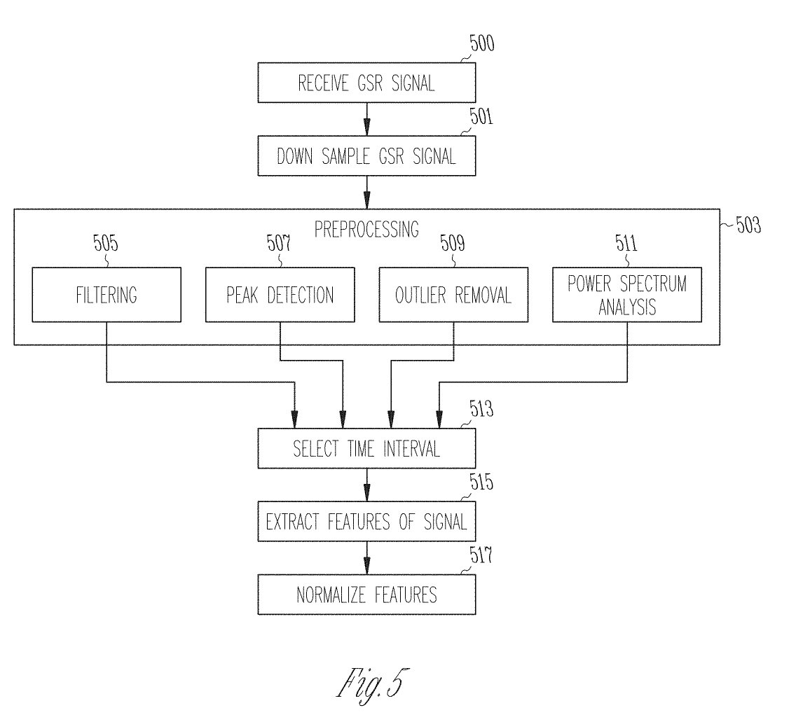

In block 307, the timing of the heartbeat indications is input to the neural network 231 for stress classification. The stress classification method is subsequently discussed in greater detail with reference to FIGS. 5 and 6.

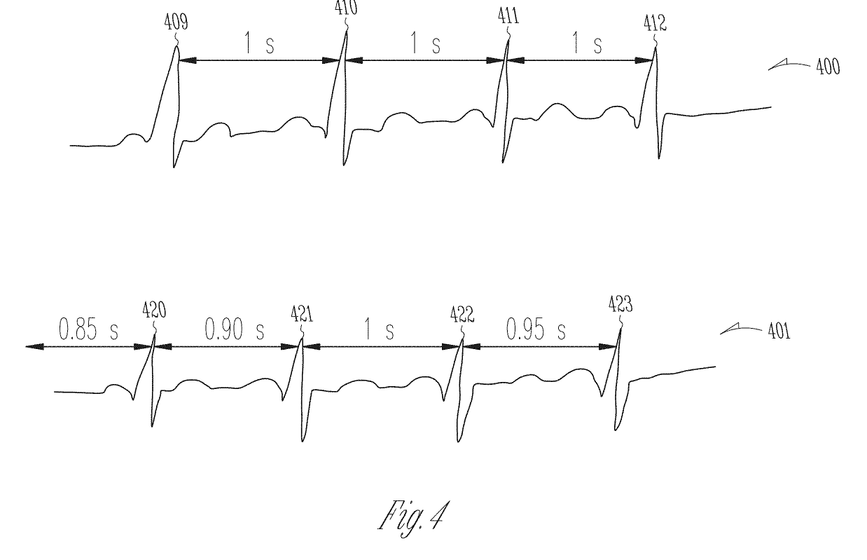

FIG. 4 illustrates various heart rate variabilities 400, 401, according to various embodiments. This figure shows two heart rate signals 400, 401. Each heart rate signal 400, 401 comprises a plurality of features 409-412, 420-423 (e.g., heartbeat indications).

The heart rate signal 400 shows a constant time period of 1 sec between adjacent heartbeat indications 410, 411. The constant time period is an indication of low HRV. The heart rate signal 401 shows a variable time period between adjacent heartbeat indications 420-423. The highly variable time periods are an indication of high HRV.

FIG. 5 illustrates a flowchart of a method for signal processing and extraction of GSR features (e.g., parameters), according to various embodiments. This method may be executed by the GSR sensor 234, the sensor subsystem 232, or both.

In the illustrated embodiment, the step of digitizing the GSR sensor data is not shown in the flowchart since it can be performed at any part of the method. For example, the entire method may be performed on a digitized GSR signal after receipt of an analog GSR signal from the sensor probes 110, 111, or the digitization may be performed anywhere between any operations.

In block 500, the GSR signal is received from the GSR sensor probes 110, 111. In block 501, the GSR signal is down sampled, also referred to as decimation, to reduce the data rate of the signal, thus reducing the size of the received data frames.

In block 503, a number of pre-processing steps are performed to extract the features (e.g., GSR peaks) from the received signal. These pre-processing steps 505, 507, 509, 511 split the GSR peak into its SCR and SCL components. For example, a filtering block 505 may be a low pass filter (LPF) at 5 Hertz (Hz) to filter out noise. After the noise has been filtered, a peak detection block 507 determines when the GSR peak occurs. In block 509, the outlier peaks are removed. In block 511, a power spectrum analysis is performed on the resulting GSR peak signal to produce a spectrum that describes the distribution of power into frequency components composing the GSR signal. According to Fourier analysis, any physical signal can be decomposed into a number of discrete frequencies or a spectrum of frequencies over a continuous range. The power spectrum analysis may be used to compute various GSR parameters (e.g., SCR frequency).

The outlier peaks may occur when the user's GSR spikes due to some unknown cause such as a surprise or other short-lived emotional response. The outlier peaks are relatively short in duration as compared to a stress GSR response.

The outputs of the blocks 505, 507, 509, 511 of the preprocessing 503 are input to a time interval selection block 513. The time interval selection block 513 determines the various time intervals of the GSR signal as illustrated in FIG. 6. The time intervals in relation to the amplitude of the GSR signal are used to extract the various SCR and SCL features of the GSR signal, in block 515. These features may then be normalized in block 517 by the application of a constant amount of gain to the GSR features to bring the average or peak amplitude to a target level (e.g., the norm). Because the same amount of gain is applied across the entire signal, the signal-to-noise ratio and relative dynamics are unchanged.

FIG. 6 illustrates parameters of the GSR signal, according to various embodiments. The use of each of these parameters in GSR signal analysis are well known in the medical art in determining stress and are not discussed in detail herein.

The SCR latency (SCR LAT) is the latency of the response onset. The SCR amplitude (SCR AMP) is the peak of the GSR curve. The SCR rise time (SCR RIS.T) is the time period over which the GSR curve rises to the peak. The SCR half-time of the recovery (SCR REC ½) is the time period from the peak of the GSR curve to the 50% signal amplitude point on the recovery side of the curve (e.g., downside after the peak).

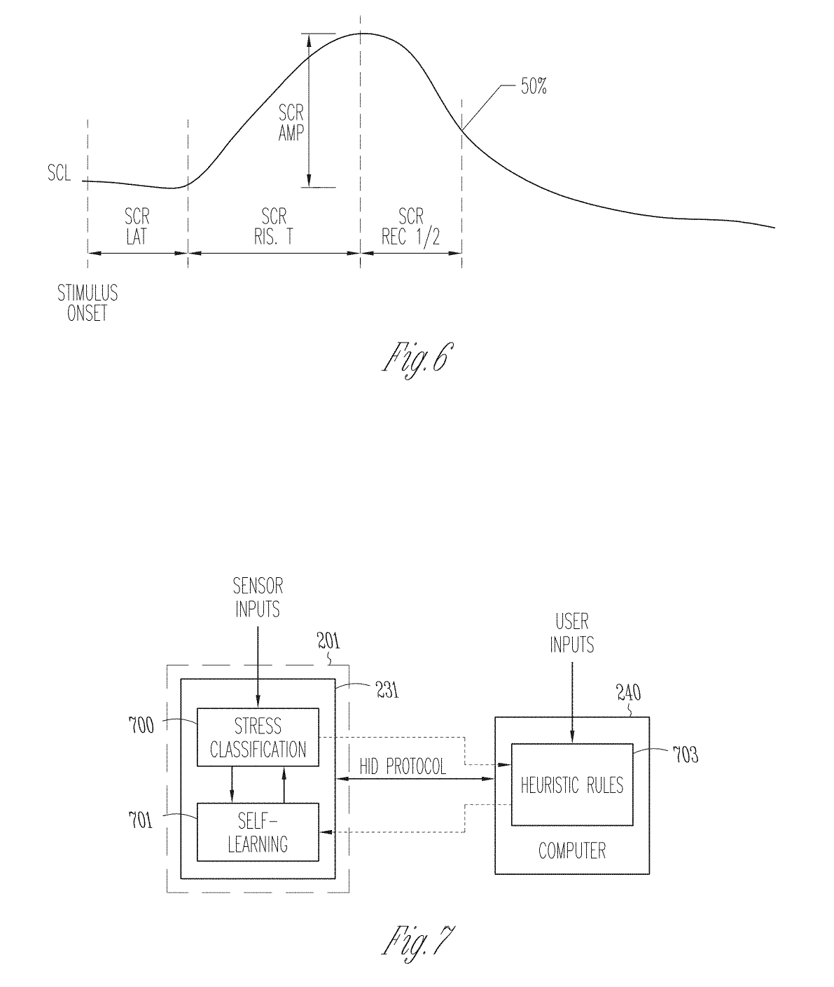

FIG. 7 illustrates a block diagram of the artificial neural network 231 in a self-learning embodiment, according to various embodiments. For purposes of illustration only, the heuristic rules 703 are shown as being stored in the computer 240. Another embodiment may include the heuristic rules 703 in the stress detection portion 201 of the human interface device 250. The heuristic rules may be defined as methods based on prior experience with the particular user using the human interface device 250. The rules are used to correct the stress classification by taking inputs from the user during self-learning operations.

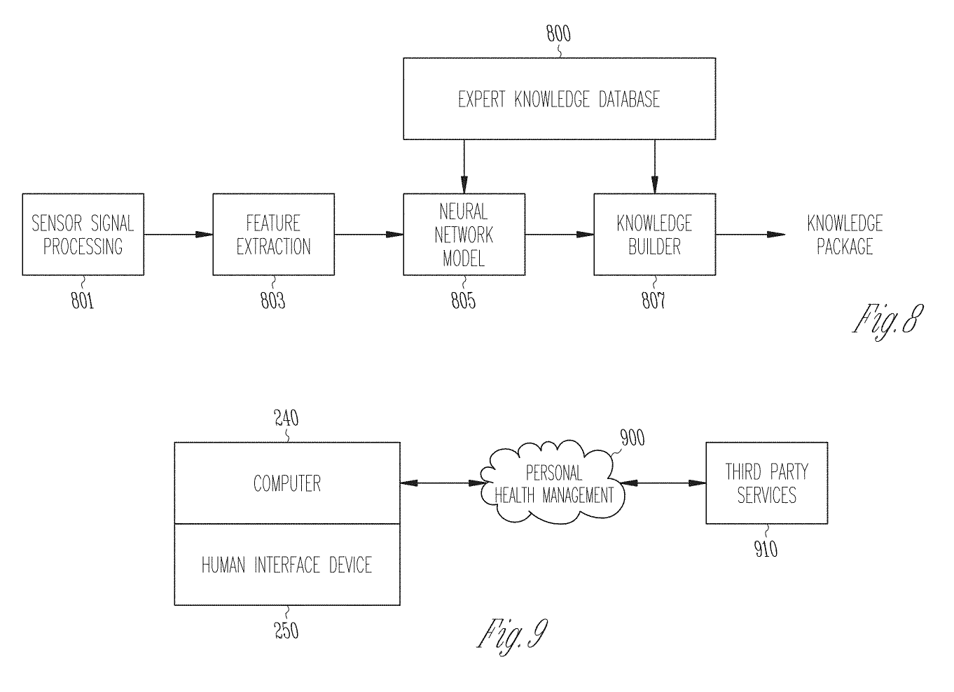

The neural network 231 of the human interface device 250 of FIGS. 1 and 2 includes a stress classification block 700 that communicates with a self-learning block 701. The stress classification block 700 is operably coupled to the ECG and GSR sensors 233, 234, through the sensor subsystem 232, of FIG. 2. The stress classification 700 in the neural network 231 is pre-trained as illustrated in FIG. 8. The computer 240 stores heuristic rules 703 and user inputs that are used by the neural network 231 to adapt to the user's ever changing stress levels and stress classification changes.

Human interface device protocol is also exchanged between the human interface device 250 and the computer 240. This protocol relates to the operation of the computer mouse and is well known in the art.

The artificial neural network 231 comprises a large number of highly interconnected processing elements (neurons) working in unison to solve a specific problem (e.g., stress classification). The neural network 231 is not programmed but instead uses an arbitrary function approximation mechanism that “learns” from observed data after a certain amount of pre-training. The goal of the neural network 231 is to solve problems in the same way that the human brain would. Using the ability to derive meaning from complicated or imprecise data, the neural network 231 extracts patterns and detects trends that are too complex to be noticed by either humans or other computer techniques.

The neural network 231 are pre-trained prior to use with generic indications of stress based on ECG and GSR. The pre-trained neural network may be based on medical data as a baseline for ECG and GSR indications of an average person. However, not everyone has the same ECG and GSR reactions to stress as the average person. Thus, the initial pre-trained neural network should be updated (e.g., retrained) to fit the person using the human interface device 250 with the self-learning mechanism based on heuristic rules.

For example, certain individuals may have a relatively low HRV or GSR parameters that may indicate stress in an average person but the user may not be stressed. That user may then indicate the lack of stress to the neural network using a Windows or other user interface on the computer to indicate that he/she is not currently experiencing stress. The neural network may be retrained for the baseline user stress response by comparing the user response and the stress classification using the heuristic rules so that the current ECG and GSR parameters are re-assigned as the baseline ECG and GSR parameters for that particular user. When that user's ECG and GSR parameters deviate from those new baseline parameters in the future, the user's stress condition may be re-evaluated in a substantially similar manner.

When sensor inputs are received by the neural network 231 in the human interface device 250, the stress classification 700 sends them to the heuristic rules 703 for comparison with the user inputs and to the self-learning 701 for subsequent learning whether those particular sensor inputs indicate stress. The heuristic rules 703 may be used to generate an initial indication on the computer to the user that stress is indicated by his or her sensor inputs (e.g., stress bar on display, pop-up window on display). The user may then input user inputs to the computer 240 and the heuristic rules block 703 whether the user is actually under stress.

If the user is not experiencing stress, the heuristic rules 703 forwards indications of the user inputs to the self-learning 701 so that the self-learning 701 no longer associates a stress condition with those particular sensor inputs. The self-learning module will then update the stress classification to reflect the discrepancy. The user may respond by clicking in a dialog box or verbally telling the computer that he/she is not stressed.

If the user is experiencing stress, they may or may not respond to the computer indication requesting confirmation that the user is experiencing stress. In either case, the self-learning 701 does not need updating since these blocks 701, 703 already associate stress with those particular sensor inputs.

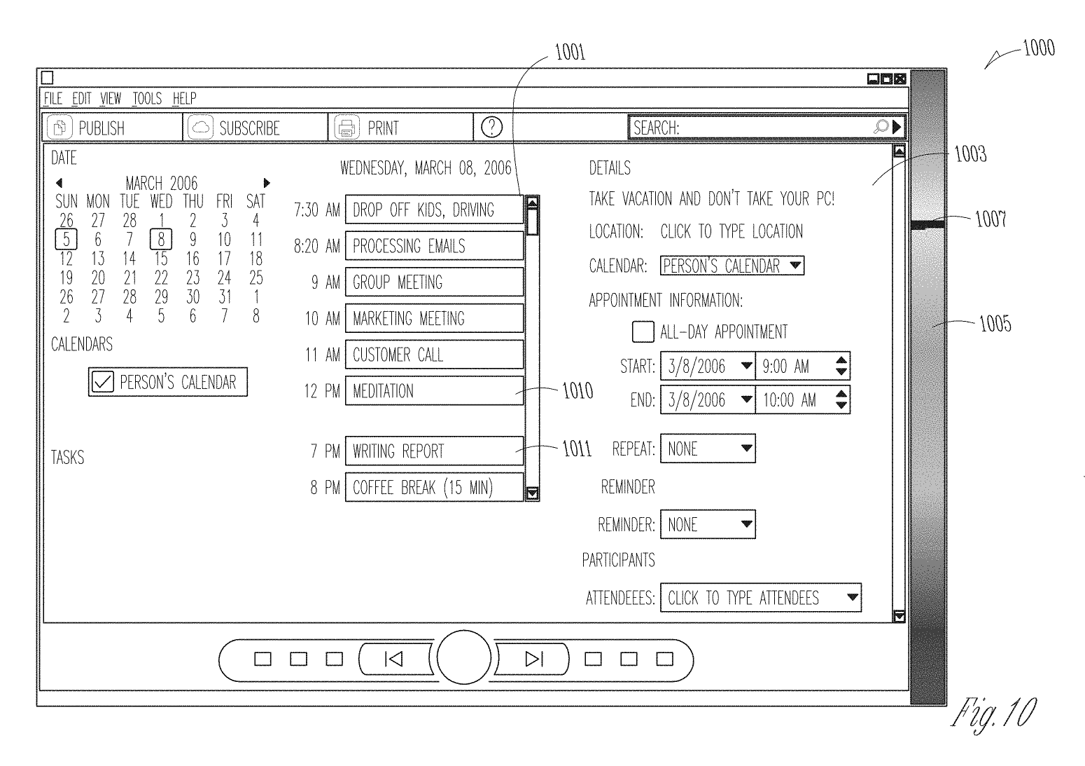

The above-described retraining of the pre-trained neural network 231 is shown taking place between the computer 240 and the stress detection portion 201 of the human interface device 250. Other embodiments may perform this retraining between the human interface device 250 and cloud servers executing a stress detection and management process or between a combination of the computer 240 and the cloud servers. Yet another embodiment may include a third party service (e.g., medical clinic) in the retraining process. Such embodiments are illustrated in FIG. 9 and discussed subsequently.

FIG. 8 illustrates a flow diagram of a method for pre-training the neural network 231, according to various embodiments. This pre-training method may be executed in cloud servers and the results downloaded to the human interface device 250 to pre-train the neural network 231. In another embodiment, the pre-training method may be executed during manufacture of the human interface device 250 so that the device 250 is pre-trained prior to purchase by the end user. In yet another embodiment, various portions of the pre-training method may be divided up between execution by cloud servers and execution elsewhere.

The method includes an expert knowledge base 800 that stores or has access to medical data on stress detection and management. For example, the expert knowledge database 800 may include a plurality of different levels ECG indications, a plurality of different levels of GSR indications, and a plurality of various combinations of ECG and GSR indications, each of these ECG, GSR, and ECG/GSR combinations may be associated with a level of stress.

In block 801, pre-training sensor input signals are input to sensor signal processing 801. The pre-training sensor input signals may include various signals having a voltage range within a typical human response for ECG and GSR indications. The voltage range may include typical human ECG and GSR voltages for a person going from a relaxed condition to a highly stressed condition.

The sensor signal processing 8001 may include analog-to-digital conversion (ADC) as well as any filtering, normalization, or other processing necessary to convert the signals to a useable form. The filtering and other processing may be performed prior to digitizing the signals using analog components or after the ADC using digital processing techniques.

The digitized sensor signals are input to feature extraction 803 that extracts the various parameters of the ECG and GSR signals. One embodiment of such parameters is discussed previously.

The extracted parameters are input to a neural network model 805 that substantially replicates the artificial neural network 231 used in the human interface device 250. The data from the knowledge database 800 is also input to the neural network model 805. The extracted features are compared to the data from the knowledge database 800, in the knowledge builder 807, in order to generate a knowledge package that is installed in the stress detection portion 201 of the human interface device 250. Since the model 805 is substantially similar to the human interface device neural network 231, the output from the model 805 will be substantially similar to any results from the human interface device's neural network 231.

FIG. 9 illustrates a block diagram of a stress management system, according to various embodiments. The computer 240 and/or the human interface device 250 of FIG. 2 may transmit stress indication data that includes the stress classification, the ECG and GSR sensor data, or both to a personal health management server in the cloud. The personal health management methods may include transmitting the stress data to third party services 910 for analysis. For example, the third party services may include medical professionals that review the data and report back to the computer/human interface device the analysis with a suggested course of action. The computer 240 may be executing a stress management tool that interfaces with the personal health management server and/or the third party services 910 to provide an indication to the user as well as one or more suggested actions to reduce or eliminate the measured stress.

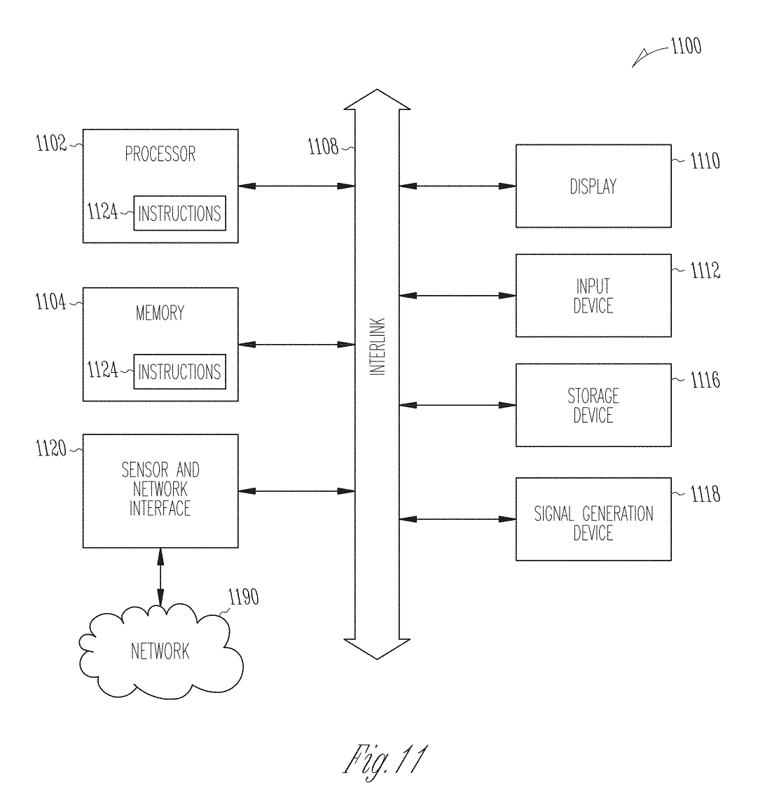

FIG. 10 illustrates a diagram of the stress management tool, according to various embodiments. The stress management tool includes a display 1000 that may be a dedicated stress indication and management display or the tool may integrate its output with other applications running on the computer 240. For example, as illustrated in FIG. 10, a Windows® One Calendar program, Outlook®, or any other computer program that may execute on the computer 240 may integrate the stress detection and management tool output into one or more displays 1000.

The illustrated display 1000 shows a column for the user's appointments 1001 for the day. Most of the appointments have been entered by the user. However, if the user has been determined to be under stress, as indicated by the stress detection classification described previously, the stress management tool may generate one or more suggested appointments 1010, 1011 to the user and place them in the user's column of appointments 1001. The suggested appointments include stress reduction activities such as meditation 1010 or a coffee break 1011. The suggested appointments 1010, 1011 may be a different color or font to alert the user to their suggested status. The user may select to keep those suggested appointments or delete them.

The stress management tool may also fill in various other fields 1003 of the display 1000 based on the stress detection classification. For example, the details field 1003 of a selected appointment may include text meant to reduce stress (e.g., take a vacation and leave your computer at home).

A stress bar 1005 may be located along one side of the display 1000 comprising multiple colors to indicate a range of levels of stress classifications. For example, the bottom of the stress bar 1005 may be green and then progressively become yellow for caution level stress and red for indicating a high stress condition.

A stress level indicator 1007 may then move up or down through those various colors of the stress bar 1005 based on the results of the stress classification.

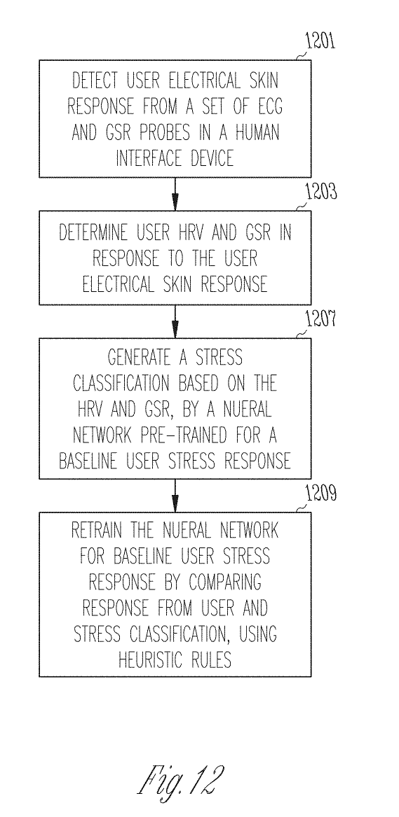

FIG. 11 illustrates a block diagram of a computer system 1100, according to various embodiments. The system 1100 may also be referred to as a computer to execute any methods disclosed herein. This block diagram may represent the computer 240, the human interface device 250, a cloud server 900, or any combination of these devices.

The system 1100 may include a processor unit 1102 (e.g., a central processing unit (CPU), a graphics processing unit (GPU), a hardware processor core, one or more processors, or any combination thereof), and memory 1104. The processor 1102 and memory 1104 together may be referred to as a controller. The various elements of the computer may communicate with each other over an interlink (e.g., bus) 1108.

The memory 1104 may include at least one transitory or non-transitory computer-readable medium on which is stored one or more sets of data structures or instructions 1124 (e.g., software) embodying or utilized by any one or more of the techniques, methods, or functions described herein. The instructions 1124 may also reside, at least partially, in additional computer-readable memories such within the hardware processor 1102 during execution thereof by the system 1100. In an example, one or any combination of the hardware processor 1102, the memory 1104 or the mass storage device 1116 may constitute non-transitory computer-readable media.

The computer 1100 may further include a display device 1110 and an alphanumeric input device 1112 (e.g., a keypad) coupled to the bus 1108. In an example, the display unit 1110 and the input device 1112 together may be a touchscreen display.

The system 1100 may additionally include a mass storage device (e.g., flash memory, random access memory (RAM), read only memory (ROM), hard disk drive (HDD), solid state drive (SSD), or any combination) 1116. A signal generation device 1118 may include a speaker. A sensor and network interface 1120 may include any wired interfaces or wireless interfaces for communication with other systems. For example, the sensor and network interface 1120 may include various radios operating using one or more radio access technologies.

The radios may operate using a Bluetooth® protocol, one or more IEEE 802.11 standards, or any other standard communicating over a wired or wireless channel with a network 1190. The network 1190 may be a peer-to-peer network, a local area network (LAN), or a wide area network (WAN) including the Internet.

FIG. 12 illustrates a flowchart of a method for stress detection and management, according to various embodiments. In block 1201, the user electrical skin response from a set of electrocardiogram (ECG) probes and a set of Galvanic skin sensor (GSR) probes is detected by a human interface device comprising mouse functions. In block 1203, a user heart rate variability (HRV) and GSR are determined in response to the user electrical skin response. In block 1207, a stress classification is generated, based on the HRV and GSR, by a neural network pre-trained for a baseline user stress response. In block 1209, the neural network is retrained for the baseline user stress response by comparing response from user to stress classification, using heuristic rules.

### Additional Notes & Examples

Example 1 is a human interface device for stress detection and management, the device comprising: a cursor control portion to generate cursor control signals; and a stress detection portion comprising: a plurality of sensor probes to detect user electrical skin response to stress; a sensor coupled to the plurality of sensor probes to generate a voltage indicative of the skin response to stress; and a neural network, coupled to the sensor, to generate a stress classification indication based on the voltage indicative of the skin response and pre-training of the neural network with a baseline stress indication.

In Example 2, the subject matter of Example 1 optionally includes wherein the plurality of sensor probes comprise a set of electrocardiogram (ECG) probes and a set of Galvanic skin sensor (GSR) probes.

In Example 3, the subject matter of any one or more of Examples 1-2 optionally includes wherein the sensor is an ECG sensor and the stress detection portion further comprises a GSR sensor coupled to the pair of GSR probes.

In Example 4, the subject matter of any one or more of Examples 1-3 optionally include wherein the wherein the neural network comprises a stress classification block coupled to a self-learning block wherein the stress classification block is configured to be pre-trained with the baseline stress indication and is coupled to the sensor.

In Example 5, the subject matter of any one or more of Examples 1-4 optionally includes wherein the self-learning block is configured to receive updates from heuristic rules based on a user response.

In Example 6, the subject matter of any one or more of Examples 1-5 optionally includes wherein the neural network is further configured to be retrained, after the pre-training, based on the user response to the baseline stress indication, using the heuristic rules.

In Example 7, the subject matter of any one or more of Examples 1-6 optionally includes wherein the stress detection portion is located on top of a mouse, on a palm rest of a computer, a back of a tablet computer, or a track pad of the computer.

In Example 8, the subject matter of any one or more of Examples 1-7 optionally includes wherein the human interface device is further configured to receive the retrained neural network based on the user response to initial stress indications using the heuristic rules.

In Example 9, the subject matter of any one or more of Examples 1-8 optionally includes wherein the stress classification block is configured to update the baseline stress indication based on the heuristic rules and the user response to the initial stress indications.

In Example 10, the subject matter of any one or more of Examples 1-9 optionally include a sensor subsystem coupled between the sensor and the neural network wherein the sensor subsystem comprises an analog-to-digital converter to generate a digital representation of the voltage indicative of the skin response.

Example 11 is a method for stress detection and management comprising: detecting a user electrical skin response from a set of electrocardiogram (ECG) probes and a set of Galvanic skin sensor (GSR) probes by a human interface device comprising cursor control functions; determining a user heart rate variability (HRV) and GSR in response to the user electrical skin response; generating a stress classification, based on the HRV and GSR, by a neural network pre-trained for a baseline user stress response; and retraining the neural network for the baseline user stress response by comparing a response from the user with the stress classification, using the heuristic rules.

In Example 12, the subject matter of Example 11 optionally includes displaying the stress classification on a computer executed stress management tool.

In Example 13, the subject matter of any one or more of Examples 11-12 optionally includes wherein displaying the stress classification comprises generating a stress bar with a stress level indicator indicative of the stress classification.

In Example 14, the subject matter of any one or more of Examples 11-13 optionally includes wherein the stress bar comprises multiple colors to indicate a range of levels of stress classifications.

In Example 15, the subject matter of any one or more of Examples 11-14 optionally include updating, in response to the stress classification, a field of a calendar program executed by a computer.

In Example 16, the subject matter of any one or more of Examples 11-15 optionally includes wherein updating the field of the calendar program comprises updating a user schedule with a suggested appointment for stress reduction.

In Example 17, the subject matter of any one or more of Examples 15-16 optionally include wherein updating the field of the calendar program comprises updating a details field for a selected appointment with text for suggested stress reduction during the selected appointment.

In Example 18, the subject matter of any one or more of Examples 11-17 optionally include transmitting the stress classification to a third party service; and receiving a suggested course of action to reduce user stress.

Example 19 is at least one computer-readable medium comprising instructions for executing stress detection and management that, when executed by a computer, cause the computer to perform any one of the method Examples of 11-18.

Example 20 is an apparatus comprising means for performing any of the methods of Examples 11-18.

Example 21 is at least one computer-readable medium comprising instructions for executing stress detection and management in a human interface device having computer mouse functions, when executed by a computer, cause the computer to: detect a user electrical skin response from a set of electrocardiogram (ECG) probes and a set of Galvanic skin response sensor (GSR) probes by the human interface device; determine a user heart rate variability (HRV) and GSR respectively in response to an ECG signal and a GSR signal generated from the user electrical skin response; generate a stress classification based on the HRV and GSR by a neural network pre-trained for the baseline user stress response; and retrain the neural network for the baseline user stress response by comparing a response from the user with the stress classification, based on the heuristic rules.

In Example 22, the subject matter of Example 21 optionally includes wherein the instructions further cause the computer to display the stress classification on a monitor coupled to the computer as part of a stress management tool.

In Example 23, the subject matter of any one or more of Examples 21-22 optionally includes wherein the instructions further cause the computer to: transmit the stress classification to a health management server; and receive a suggested course of action to reduce user stress.

In Example 24, the subject matter of any one or more of Examples 21-23 optionally include wherein the instructions further cause the computer to extract parameters from a GSR signal of the GSR sensor indicative of user stress.

In Example 25, the subject matter of any one or more of Examples 21-24 optionally includes wherein the instructions further cause the computer to extract a skin conductance response (SCR) latency, an SCR amplitude, an SCR rise time, and an SCR half-time of a recovery of the SCR.

In Example 26, the subject matter of any one or more of Examples 21-25 optionally include wherein the instructions further cause the computer to detect a user heart rate to generate the HRV.

In Example 27, the subject matter of any one or more of Examples 21-26 optionally include wherein the instructions further cause the computer to digitally process the ECG signal and the GSR signal to determine the HRV and the GSR.

Example 28 is a system for stress detection and management comprising: means for detecting a user electrical skin response from a set of electrocardiogram (ECG) probes and a set of Galvanic skin sensor (GSR) probes by a human interface device comprising cursor control functions; means for determining a user heart rate variability (HRV) and GSR in response to the user electrical skin response; means for generating a stress classification, based on the HRV and GSR, by a neural network pre-trained for a baseline user stress response; and means for retraining the neural network for the baseline user stress response by comparing a response from the user with the stress classification, using the heuristic rules.

In Example 29, the subject matter of Example 28 optionally includes means for displaying the stress classification on a computer executed stress management tool.

In Example 30, the subject matter of any one or more of Examples 28-29 optionally includes wherein the means for displaying the stress classification comprises means for generating a stress bar with a stress level indicator indicative of the stress classification.

In Example 31, the subject matter of any one or more of Examples 28-30 optionally includes wherein the stress bar comprises multiple colors to indicate a range of levels of stress classifications.

In Example 32, the subject matter of any one or more of Examples 28-31 optionally include means for updating, in response to the stress classification, a field of a calendar program executed by a computer.

In Example 33, the subject matter of any one or more of Examples 28-32 optionally includes wherein updating the field of the calendar program comprises means for updating a user schedule with a suggested appointment for stress reduction.

In Example 34, the subject matter of any one or more of Examples 28-33 optionally include wherein the means for updating the field of the calendar program comprises means for updating a details field for a selected appointment with text for suggested stress reduction during the selected appointment.

In Example 35, the subject matter of any one or more of Examples 28-34 optionally include means for transmitting the stress classification to a third party service; and means for receiving a suggested course of action to reduce user stress.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, also contemplated are examples that include the elements shown or described. Moreover, also contemplated are examples using any combination or permutation of those elements shown or described (or one or more aspects thereof), either with respect to a particular example (or one or more aspects thereof), or with respect to other examples (or one or more aspects thereof) shown or described herein.

Publications, patents, and patent documents referred to in this document are incorporated by reference herein in their entirety, as though individually incorporated by reference. In the event of inconsistent usages between this document and those documents so incorporated by reference, the usage in the incorporated reference(s) are supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to suggest a numerical order for their objects.

The above description is intended to be illustrative, and not restrictive. For example, the above-described examples (or one or more aspects thereof) may be used in combination with others. Other embodiments may be used, such as by one of ordinary skill in the art upon reviewing the above description. The Abstract is to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. However, the claims may not set forth every feature disclosed herein as embodiments may feature a subset of said features. Further, embodiments may include fewer features than those disclosed in a particular example. Thus, the following claims are hereby incorporated into the Detailed Description, with a claim standing on its own as a separate embodiment. The scope of the embodiments disclosed herein is to be determined with reference to the appended claims, along with the full scope of equivalents to which such claims are entitled.

What is claimed is:

1. A human interface device for stress detection and management, the device comprising:
a cursor control portion to generate cursor control signals; and
a stress detection portion comprising:
a plurality of sensor probes to detect user electrical skin response to stress;
a sensor coupled to the plurality of sensor probes to generate a voltage indicative of the skin response to stress; and
a neural network, coupled to the sensor, to generate a stress classification indication based on the voltage indicative of the skin response and pre-training of the neural network with a baseline stress indication.

2. The human interface device of claim 1, wherein the plurality of sensor probes comprise a set of electrocardiogram (ECG) probes and a set of Galvanic skin sensor (GSR) probes.

3. The human interface device of claim 2, wherein the sensor is an ECG sensor and the stress detection portion further comprises a GSR sensor coupled to the pair of GSR probes.

4. The human interface device of claim 1, wherein the neural network comprises a stress classification block coupled to a self-learning block wherein the stress classification block is configured to be pre-trained with the baseline stress indication and is coupled to the sensor.

5. The human interface device of claim 4, wherein the self-learning block is configured to receive updates from heuristic rules based on a user response.

6. The human interface device of claim 5, wherein the neural network is further configured to be retrained, after the pre-training, based on the user response to the baseline stress indication, using the heuristic rules.

7. The human interface device of claim 6, wherein the stress detection portion is located on top of a mouse, on a palm rest of a computer, a back of a tablet computer, or a track pad of the computer.

8. The human interface device of claim 7, wherein the human interface device is further configured to receive the retrained neural network based on the user response to initial stress indications using the heuristic rules.

9. The human interface device of claim 8, wherein the stress classification block is configured to update the baseline stress indication based on the heuristic rules and the user response to the initial stress indications.

10. The human interface device of claim 1, further comprising a sensor subsystem coupled between the sensor and the neural network wherein the sensor subsystem comprises an analog-to-digital converter to generate a digital representation of the voltage indicative of the skin response.

11. A method for stress detection and management comprising:
detecting a user electrical skin response from a set of electrocardiogram (ECG) probes and a set of Galvanic skin sensor (GSR) probes by a human interface device comprising cursor control functions;
determining a user heart rate variability (HRV) and GSR in response to the user electrical skin response;
generating a stress classification, based on the HRV and GSR, by a neural network pre-trained for a baseline user stress response; and
retraining the neural network for the baseline user stress response by comparing a response from the user with the stress classification, using the heuristic rules.

12. The method of claim 11, further comprising displaying the stress classification on a computer executed stress management tool.

13. The method of claim 12, wherein displaying the stress classification comprises generating a stress bar with a stress level indicator indicative of the stress classification.

14. The method of claim 13, wherein the stress bar comprises multiple colors to indicate a range of levels of stress classifications.

15. The method of claim 11, further comprising updating, in response to the stress classification, a field of a calendar program executed by a computer.

16. The method of claim 15, wherein updating the field of the calendar program comprises updating a user schedule with a suggested appointment for stress reduction.

17. The method of claim 15, wherein updating the field of the calendar program comprises updating a details field for a selected appointment with text for suggested stress reduction during the selected appointment.

18. The method of claim 11, further comprising:
transmitting the stress classification to a third party service; and
receiving a suggested course of action to reduce user stress.

19. At least one computer-readable medium comprising instructions for executing stress detection and management in a human interface device having computer mouse functions, when executed by a computer, cause the computer to:
detect a user electrical skin response from a set of electrocardiogram (ECG) probes and a set of Galvanic skin response sensor (GSR) probes by the human interface device;
determine a user heart rate variability (HRV) and GSR respectively in response to an ECG signal and a GSR signal generated from the user electrical skin response;
generate a stress classification based on the HRV and GSR by a neural network pre-trained for the baseline user stress response; and
retrain the neural network for the baseline user stress response by comparing a response from the user and the stress classification, based on the heuristic rules.

20. The computer-readable medium of claim 20, wherein the instructions further cause the computer to display the stress classification on a monitor coupled to the computer as part of a stress management tool.

21. The computer-readable medium of claim 19, wherein the instructions further cause the computer to:
transmit the stress classification to a health management server; and
receive a suggested course of action to reduce user stress.

22. The computer-readable medium of claim 19, wherein the instructions further cause the computer to extract parameters from a GSR signal of the GSR sensor indicative of user stress.

23. The computer-readable medium of claim 22, wherein the instructions further cause the computer to extract a skin conductance response (SCR) latency, an SCR amplitude, an SCR rise time, and an SCR half-time of a recovery of the SCR.

24. The computer-readable medium of claim 20, wherein the instructions further cause the computer to detect a user heart rate to generate the HRV.

25. The computer-readable medium of claim 20, wherein the instructions further cause the computer to digitally process the ECG signal and the GSR signal to determine the HRV and the GSR.
